(19) United States
(12) Patent Application Publication
(10) Pub. No.: US 2021/0228648 A1
(43) Pub. Date: Jul. 29, 2021

(54) WORM PRODUCTS AND METHODS OF USE

(71) Applicant: Aarhus Universitet, Aarhus C (DK)

(72) Inventors: Peter Nejsum, Brabrand (DK); Martin Roelsgaard Jakobsen, Risskov (DK); Sidsel Dahl Andersen, Aarhus C (DK); Christian Krapp, Ebeltoft (DK)

(73) Assignee: Aarhus Universitet, Aarhus C (DK)

(21) Appl. No.: 15/734,650

(22) PCT Filed: Jun. 4, 2019
(86) PCT No.: PCT/EP2019/064466

§ 371 Date: Dec. 3, 2020

(30) Foreign Application Priority Data
Jun. 4, 2018 (EP) 18175721.2

(51) Int. Cl. A61K 35/62; A61P 37/06

(52) U.S. Cl. CPC A61K 35/62; A61P 37/06

(57) ABSTRACT

The present invention relates to the use of a composition comprising a helminth product for prevention, treatment and/or amelioration of a clinical condition associated with the RIG-LISTING and TLR pathways. Specifically, the composition is provided for use in the treatment of clinical conditions associated with constitutive activation of type I interferon (IFN), including monogenic type I interferonopathies and inflammatory or autoimmune disorders. The present invention also relates to different methods of manufacturing a helminth product.

### TECHNICAL FIELD

The present invention relates to helminth products and their use in the prevention, treatment and/or amelioration of clinical conditions associated with the RIG-I/STING and TLR pathways. Specifically, helminth products are provided for use in the treatment of clinical conditions associated with constitutive activation of type I interferon (IFN), including monogenic type I interferonopathies and inflammatory or auto-immune disorders. In addition, helminth products are also provided for use in the treatment of clinical conditions associated with decreased or limited activation of the innate immune system, including inflammatory disorders and cancer.

### BACKGROUND

Stimulus-dependent activation of immune responses is essential to achieve optimal tempo-spatial immune activities at sites of infection and to avoid tissue damage. The innate immune system relies on several groups of pattern recognition receptors (PRRs) that recognize viral nucleic acids. These PRRs include the sensors cyclic GMP-AMP Synthase (cGAS) and retinoic acid-inducible gene I (RIG-I) which recognize different nucleic acids in the cytosol. Once activated, they induce different signaling pathways with partly overlapping signaling molecules, leading to the production of a variety of innate immune inflammatory molecules, including type I-Ill interferons, cytokines, chemokines and interleukins. cGAS senses the aberrant presence and concentration of DNA in the cytosol, leading to the formation of cyclic GMP-AMP (cGAMP). cGAMP then binds to the endoplasmic reticulum-bound protein stimulator of interferon genes (STING). STING then traffics from ER to an ER-GOLGI intermedium complex where it recruits the kinase TBK1, and initiates phosphorylation of STING, TBK1 and subsequently the transcription factor IRF3. In parallel, activation of STING do also lead to signaling through the NF-kB pathway.

RIG-I detects viral RNAs and then binds to mitochondrial antiviral signaling protein (MAVS) Once engaged, MAVS signaling activates three kinases that serve as regulators of inflammation, Inhibitor of κB-Kinase (IKK)-γ, TKB1 and IKK-ε. These kinases phosphorylate IRF-1,-3, -7, and Nuclear Factor (NF)-κB.

Importantly, IFNs and other pro-inflammatory cytokines produced in response to cGAS/STING and RIG-I/MAVS activation drive a feed-forward signaling loop that maintains high expression levels of these factors, IFNs and additional pro-inflammatory IFN-stimulated genes (ISGs), by maintaining phosphorylation and activation of the transcription factors and by phosphorylation of the transcription factor Signal Transducer and Activator of Transcription (STAT)-1, which occurs in response to IFN-α/β receptor (IFNAR)-mediated activation of JAK-STAT signaling.

The pathway that leads to IFN activation has been extensively studied both in terms of the proteins binding cytosolic RNA/DNA and those needed for subsequent downstream signaling and immune activation. Localization of nucleic acids to the cytosol is associated with tumorigenesis or viral infection, hence, the induction of type I IFNs by RNA or DNA is essential for defense against virus infections and for anti-cancer immunity.

Another important PRR for antimicrobial sensing is Toll-like receptors, including TLR3, 4, 7, 8, and 9. They are involved in sensing both viral nucleic acids and bacterial infections. For example, TLR4 recognizes lipopolysaccharide (LPS)—a major component of the outer membrane of Gram-negative bacteria. Very limited amounts of LPS released from bacterial infections can initiate potent innate immune responses that prime the immune system against further infection. However, when the LPS response is not properly controlled it can lead to fatal septic shock syndrome.

Negative regulation of immune pathways is, essential to achieve resolution of immune responses and to avoid excess inflammation. Pathological roles have been ascribed to type I IFNs in bacterial infections and in chronic viral infections. Most notably, excess activation of the RIG-I/STING pathway is associated with clinical conditions involving constitutive activation the Nf-KB pathway as well as Interferons, including monogenic type I interferonopathies and inflammatory or auto-immune disorders. Also, LPS is associated with sepsis under bacteremia.

There is an increasing body of evidence that people living in areas of the world where soil transmitted helminths (worms) are less prone to chronic immunopathological diseases than people in non-endemic countries. This has been linked to a modern lifestyle (‘hygiene hypothesis’), and in Europe and North America autoimmune diseases affect about 5% of the population.

Inflammatory disorders are associated with an inappropriate immunological response towards auto-antigens or allergens, and as worms induce anti-inflammatory and/or regulatory immunological responses, they may mediate beneficial immunomodulatory effects in the host. In this way, it has been shown that helminths suppress inflammatory responses in macrophages and dendritic cells, and induce regulatory T-cells in animal models of inflammatory diseases, consistent with the notion of helminths inducing a modified Th2-type response and suppressing pro-inflammatory Th1-type responses. However, the underlying mechanisms behind this immune-modulation remain elusive.

### SUMMARY

Due to the central role of TLR, RIG-I and STING in regulating the level of IFN and cytokine expression under innate immune responses, methods of modulating these pathways are highly desirable in order for preventing, treating and/or ameliorating disorders associated with constitutive activation of type I IFN or NF-kB pathway (see FIG. 1).

The inventors have surprisingly identified that helminth products are capable of modulating the TLR/RIG-I/STING pathways and thereby modulate the NF-kB driven and IRF3 driven inflammatory responses induced by these pathways. The helminth products thus provide promising avenues for preventing, treating and/or ameliorating disorders associated with the various innate immune pathway.

Thus, in one aspect, the present invention provides a composition comprising a helminth product for use in preventing, treating and/or ameliorating a clinical condition associated with the RIG-I/STING pathway.

In a second aspect, the present invention provides a composition for use in inhibiting the RIG-I/STING pathway in an individual, the method comprising administering a helminth product or fraction thereof to said individual.

In a third aspect, the present invention provides a composition for use in inducing the RIG-I/STING pathway in an individual, the method comprising administering a helminth product or fraction thereof to said individual.

In a fourth aspect, the present invention provides a composition for use in preventing, treating and/or ameliorating a clinical condition associated with the TLR pathways.

In a fifth aspect, the present invention provides a composition for use in inhibiting the TLR pathway in an individual, the method comprising administering a helminth product or fraction thereof to said individual.

In a sixth aspect, the present invention provides a composition for use in inducing the TLR pathway in an individual, the method comprising administering a helminth product or fraction thereof, to said individual.

### DESCRIPTION OF DRAWINGS

FIG. 1

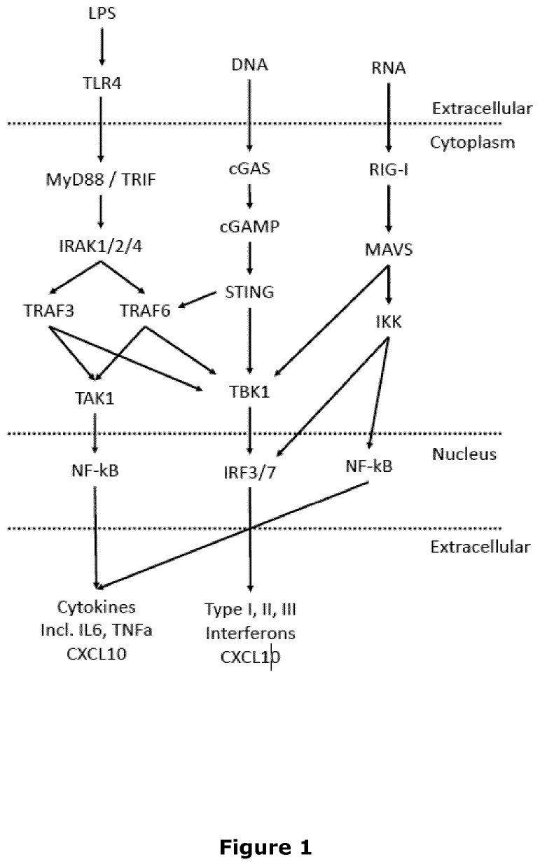

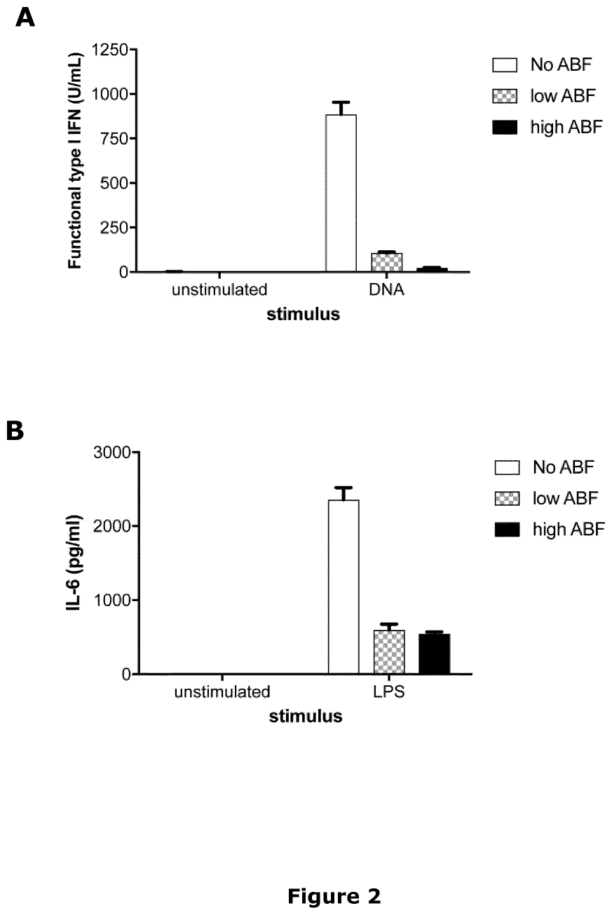

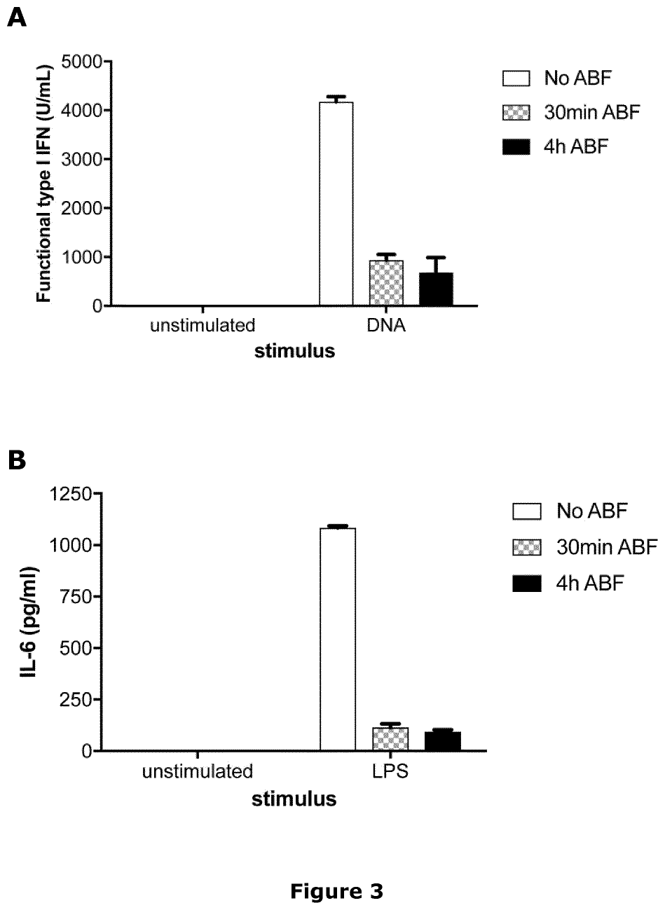

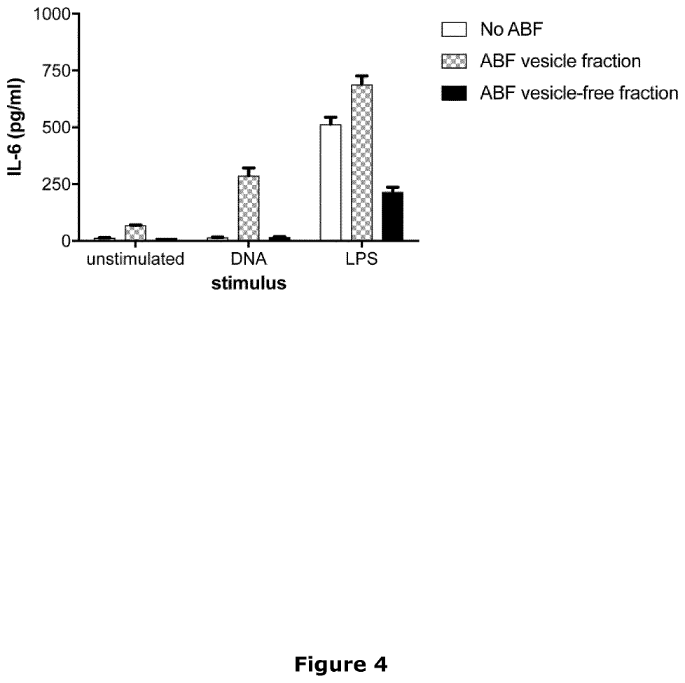

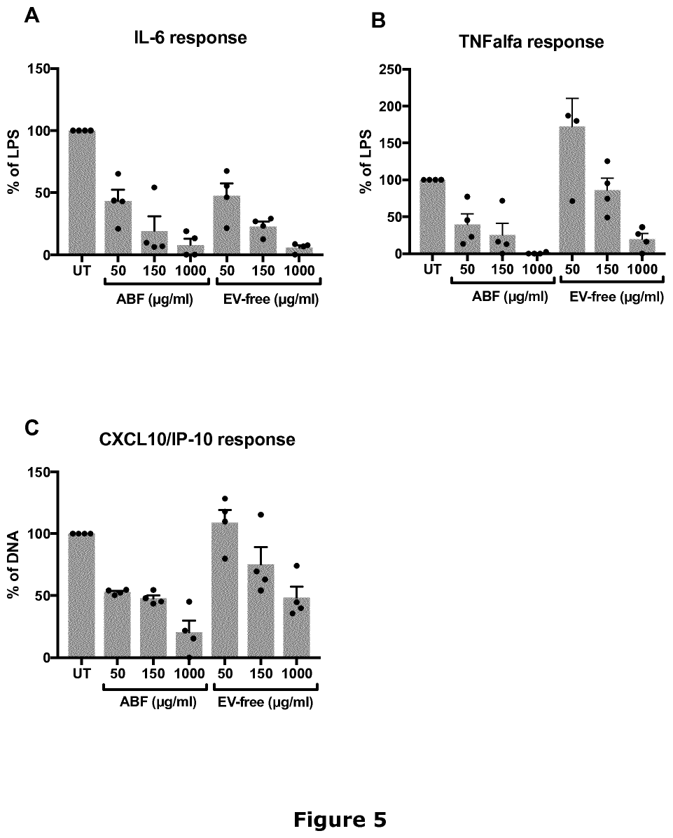

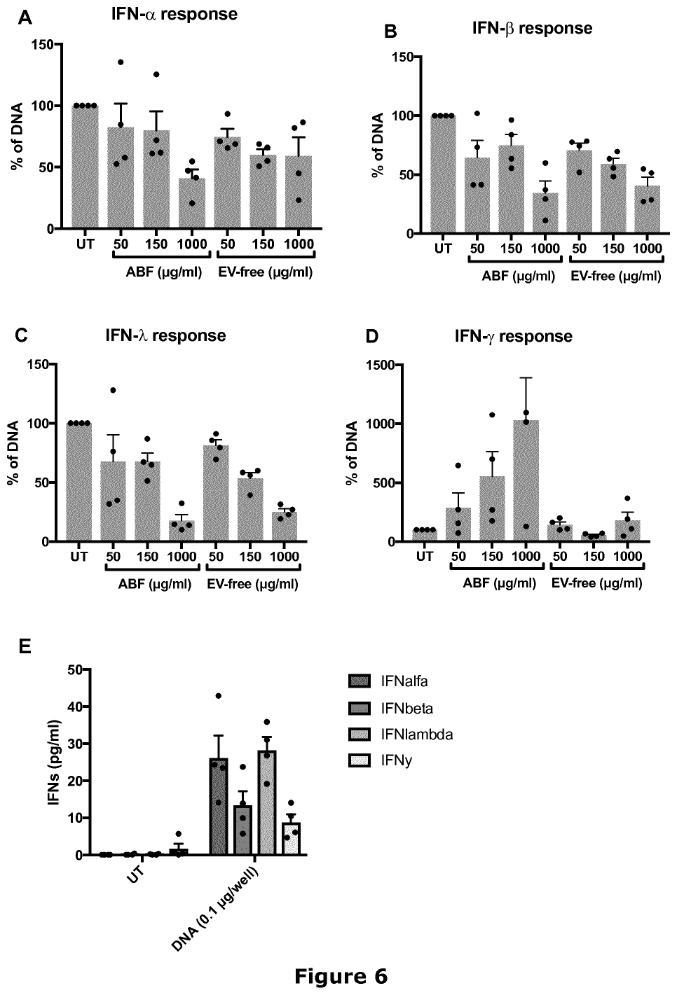

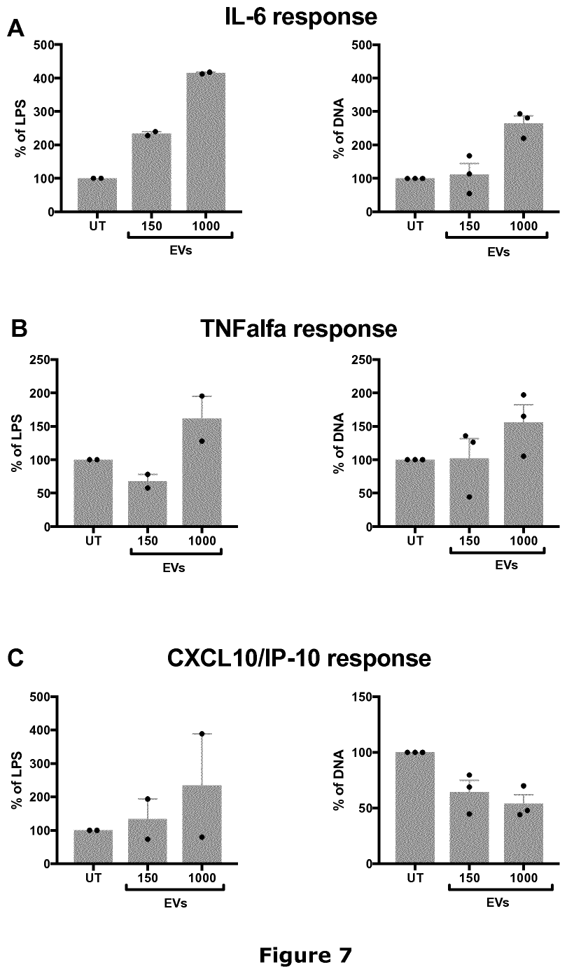

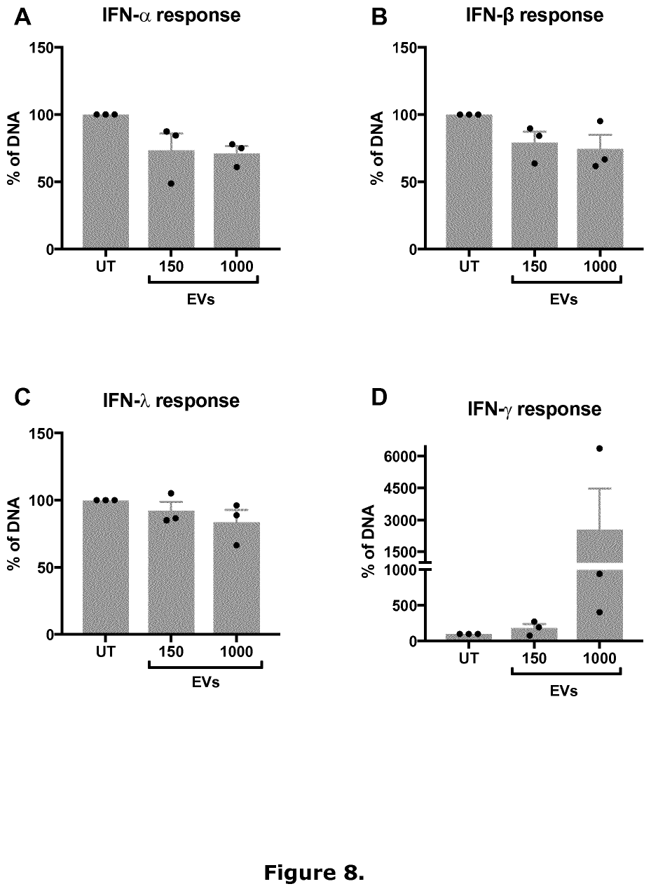

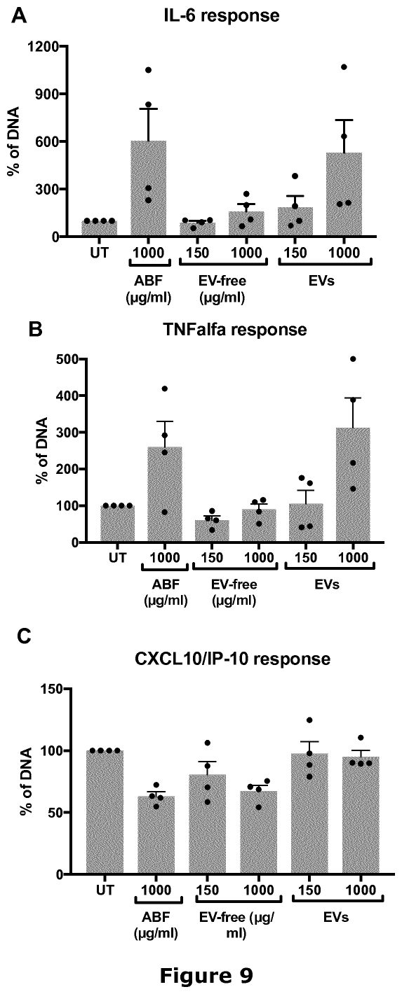

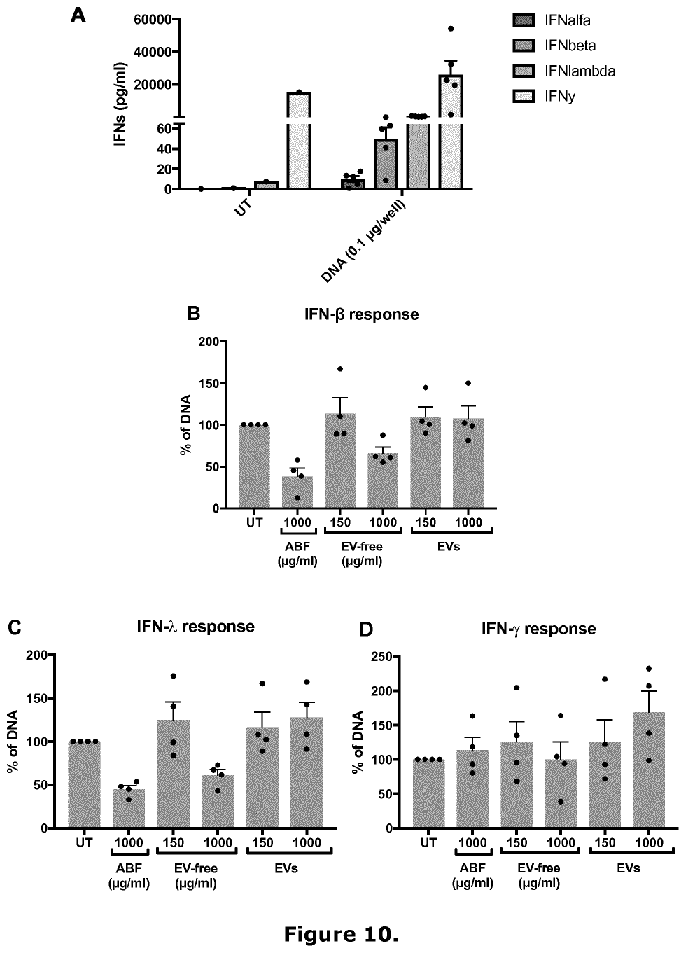

A simplified depiction of the pathways targeted and investigated in the following experiments.

LPS is detected extracellularly by Toll-like-receptor 4 (TLR4), which then activates TNF-receptor-associated-factor 6 (TRAF6) via Myeloid-differentiation-primary-response 88 (MyD88). TRAF6 then activates nuclear factor ‘kappa-light-chain-enhancer’ of activated B-cells (NfκB), which moves into the nucleus and acts as a transcription factor for pro-inflammatory genes. This then leads, among other things to the release of a series of cytokines and interleukins such as TNFα and IL6. This pathway is conserved for most Toll-like receptors.

Foreign DNA in the cytoplasm (in our experiments delivered via liposomal transfection) is detected by cyclic-GMP-AMP-synthase (cGAS) with the support of gamma-interferon-inducible protein (IF116). cGAS then produces a second messenger molecule which activates stimulator-of-interferon-genes (STING), which in turn activates serine/threonine-protein kinase (TBK1). TBK1 then phosphorylates the transcription factor interferon-regulatory-factor 3 (IRF3) as well as NF-kB, which then are translocated to the nucleus, leading to transcription of both antiviral type I, II, and III interferons (IFNs) and pro-inflammatory cytokines.

FIG. 2

IFN-I response and IL-6 response in differentiated THP-1 cells after pre-treatment with varying amounts of Ascaris suum adult body fluids (ABF). The amounts of ABF used for the treatment were 600 μg protein per 5×10⁴ cells of ABF (high ABF) and 350 μg per 5×10⁴ cells of ABF (low ABF). Treatment of differentiated THP-1 cells with A. suum ABF resulted in reduced IFN-1 and IL-6 response in a concentration dependent manner, upon stimulation with DNA (STING agonist) or LPS (TLR-4 ligand).

FIG. 3

IFN-I response and IL-6 response in differentiated THP-1 cells after pre-treatment with Ascaris suum adult body fluids (ABF) at varying time periods, 30 min or 4 h, followed by DNA stimulation. Treatment of differentiated THP-1 cells with A. suum ABF resulted in reduced IFN-I and IL-6 response upon stimulation with DNA (STING agonist) or LPS (TLR-4 ligand), with increased duration of treatment resulting in increased efficacy.

FIG. 4

IL-6 response in differentiated THP-1 cells after pre-treatment with different fractions of Ascaris suum adult body fluids (ABF). The A. suum fractions used for the treatment were prepared by a series of centrifugation and ultra-centrifugations steps and consist of ABF vesicle-enriched fraction (ABF vesicle fraction) and ABF vesicle-depleted fraction (ABF vesicle-free fraction). Treatment of differentiated THP-1 cells with the A. suum ABF vesicle-depleted fraction resulted in different effects on the IL-6 production. The ABF vesicle-enriched fraction increases the IL-6 response to DNA (STING agonist) and LPS (TLR-4 ligand). The ABF vesicle-depleted fraction decreases the IL-6 response after LPS stimulation.

FIG. 5

The immunological effects in Peripheral Blood Mononuclear Cells (PBMCs) isolated from healthy donors after pre-treatment with varying amount of Ascaris suum ABF and extracellular vesicle (EV)-depleted fractions (EV-free). EV-depleted fractions used for treatment was prepared by a series of centrifugation and ultra-centrifugations step (see method).

Treatment of PBMCs with A. suum ABF and EV-depleted fraction resulted in reduced IL-6 and TNF-alpha response upon stimulated with LPS (TLR-4 ligand) in a concentration dependent manner (FIG. 5A+B). Treatment of PBMCs with A. suum ABF and EV-depleted fraction also reduced CXCL10/IP-10 response in a concentration dependent manner upon stimulation with DNA (STING agonist) (FIG. 5C).

Data is expressed as a percentage of the cytokine secretion when compared to LPS or DNA stimulation alone. Data are shown as mean with SEM.

FIG. 6

The IFNs responses in PBMCs isolated from healthy donors after pre-treatment with varying amounts of Ascaris suum ABF and then stimulated with DNA (STING agonist). Treatment of PBMCs with A. suum ABF and EV-depleted fraction (EV-free) resulted in reduced type I and type II IFNs (FIG. 6A-C). In contrary, type II IFN responses was significantly increased by ABF, however not by the EV-depleted fractions, in concentration dependent manners (FIG. 6D).

Data is expressed as a percentage of the cytokine secretion when compared to DNA stimulation alone. Concentrations (pg/ml) of interferons in DNA stimulations alone can be seen in FIG. 6E. Data are shown as mean with SEM.

FIG. 7

The immunological effects in PBMCs isolated from healthy donors after pre-treatment with varying amount of an ABF EV-enriched fraction (EVs). EVs used for the treatment were prepared by a series of centrifugation and ultra-centrifugations step.

Treatment of PBMCs with an ABF EV-enriched fraction resulted in increased IL-6 and TNF-alpha response upon stimulated with LPS (TLR-4 ligand) and DNA (STING agonist) in a concentration dependent manner (FIG. 7A+B). Treatment of PBMCs with an ABF EV-enriched fraction increased CXCL10/IP-10 response upon stimulation with LPS. In contrast, treatment with the ABF EV-enriched fraction resulted in a reduced CXCL10/IP-10 response upon stimulation with DNA (FIG. 7C).

Data is expressed as a percentage of the cytokine secretion when compared to LPS or DNA stimulation alone. Data are shown as mean with SEM.

FIG. 8

The IFNs responses in PBMCs isolated from healthy donors after pre-treatment with varying amounts of an ABF EV-enriched fraction (EVs). EVs used for the treatment were prepared by a series of centrifugation and ultra-centrifugations step. Treatment of PBMCs with an ABF EV-enriched fraction did only marginally affect the responses of type I and III IFNs following stimulation with DNA (STING agonist) and was not dependent on concentration of EVs (FIG. 8A-C). On the contrary, type II IFN responses was significantly increased with the ABF EV-enriched fractions in a concentration dependent manner (FIG. 8D).

Data is expressed as a percentage of the cytokine secretion when compared to DNA stimulation alone. Data are shown as mean with SEM.

FIG. 9

The immunological responses in monocyte-derived macrophages (MDMs) isolated from healthy donor, pre-treatment with Ascaris suum ABF and varying amounts of EV-depleted (EV free) and EV-enriched fractions (EVs).

Treatment of MDMs with A. suum ABF and the EV-enriched fraction, but not EV-depleted fraction, increased the IL-6 and TNFalpha response upon stimulation with DNA (FIG. 9A+B). On the contrary, treatment of MDMs with A. suum ABF and EV-depleted fraction resulted in reduction of CXCL10/IP-10 response upon stimulated with DNA, whereas the EV-enriched fraction did not affect the response (FIG. 9C).

Data is expressed as a percentage of the cytokine secretion when compared to DNA stimulation alone. Data are shown as mean with SEM.

FIG. 10

The IFNs responses in monocyte derived macrophages (MdMs) isolated from healthy donor, pre-treatment with Ascaris suum ABF and varying amounts of EV-depleted (EV-free) and EV-enriched fractions (EVs) prior to stimulation with DNA (STING agonist). Treatment of MDMs with DNA only affected the expression of IFN β, γ and λ (FIG. 10A). Treatment of MDMs with A. suum ABF and EV-depleted fraction resulted in reduced IFN-β and -λ, response upon stimulation with DNA in a concentration dependent manner (STING agonist) (FIG. 10B+C). However, the IFN-γ response was not affected by treatment (FIG. 10D). Furthermore, treatment with the EV-enriched fraction did not affect type I and III IFNs, but increased IFN-γ response in a concentration dependent manner (FIG. 10B-D).

Data is expressed as a percentage of the cytokine secretion when compared to DNA stimulation alone. Concentrations (pg/ml) of interferons in DNA stimulations alone can be seen in FIG. 10E. Data are shown as mean with SEM.

### DETAILED DESCRIPTION

The present invention provides methods for preventing, treating and/or ameliorating a clinical condition associated with TLR or the RIG-I/STING pathway, the methods comprise administering a helminth product or fractionation thereof, or purified compound thereof, or a recombination/synthetic derivative thereof to an individual in need thereof. Since the TLR/RIG-I/STING pathways are involved in induction of IFNs and inflammatory cytokines, such methods are highly important for treatment of clinical conditions associated with constitutive activation of the immune system, including IFNs, such as monogenic type I interferonopathies and autoimmune or inflammatory disorders.

Helminths and Helminth Products

In one embodiment, the present disclosure provides helminth products for use in preventing, treating and/or ameliorating a clinical condition associated with the RIG-I/STING pathway or TLR pathway.

In one embodiment, the helminth belongs to the order Ascaridida (also called Rhabditida) of parasitic roundworms. This order includes worms having three characteristic “lips” on the anterior end. This order includes many relevant parasitic helminths of humans and domestic animals. Important families are:
the Anisakidae, known as the “marine mammal ascarids”;
the Ascarididae;
the Cosmocercidae, which include taxa that parasitize certain amphibians;
the Toxocaridae, which include parasites of canids, felids, and raccoons;
The Ascaridiidae and Heterakidae, which include roundworms of birds.

In a more preferred embodiment, the helminth belongs to the Ascaridoidea superfamily, which includes the preferred giant intestinal roundworms (Ascaris spp.). Thus, in one embodiment, the helminth is of the Ascaris genus. In a more specific embodiment, the helminth is Ascaris suum (pigs). Apart from Ascaris suum, the Ascaris genus only includes A. lumbricoides (human). It has however, been an ongoing discussion whether they represent one or two species. The present disclosure therefore relates to both A. suum and A. lumbricoides helminth products. The two species are so closely related that it is safe to assume that the derived worm products from human and pig Ascaris have similar effects on the human immune system. Ascaris sp. can occasional also be found in other host species such as chimpanzees, lambs/sheep and calves/cows. The Ascaridoidea superfamily includes a number of parasitic worms that have similar biology and lifecycle in their mammalian hosts. For example Toxocara spp. in dogs, cats, cattle as well as Parascaris equorum (also called P. univalens) in horses infect their hosts with eggs containing a third stage larvae and the worms undergo migration from the intestine, to the liver, to the lungs and further to the small intestine where they establish as adults. This is the same lifecycle as for A. suum. The present invention therefore also in more specific embodiments relates to Toxocara spp. and Parascaris equorum helminth products. In a separate embodiment, the helminth is Toxascaris spp., Ascaridia spp, Heterakis spp., Baylisascaris spp., Contracaecum spp, Pseudoterranova spp or Anisakis spp.

Helminth products are meant to include any fraction, lysate, fractionation, homogenate, recombinant/synthetic derivate, purified compound or extract of helminths. Broadly, helminth products of this invention can be divided into 1) secretion products 2) helminth homogenate 3) lysates such as worm body fluid (e.g. ABF) and extracellular vesicle-enriched fractions and EV-depleted fractions deriving from the three products.

1) Secretion products are defined as the products released by the worm(s) into a given incubation media (medium solution). This includes all the molecules released from the worms into the medium. The worm secretion products can then be isolated from the medium solution.

2) Helminth homogenate, which includes crude helminth products and soluble helminth products are defined as all molecules that can be derived from a homogenized worm or part of the worm. Worms may be homogenized in PBS using a mortar or tissue homogenizer. Soluble parasite products can be obtained by taking the homogenated worm, adding PBS and mixing, and then spinning down all larger particles. The supernatant then contains “soluble parasite products”. Instead of a hydrophilic solvent as PBS the suspension solution can also be lipophilic.

3) Lysates such as worm body fluid (BF) refers to the liquid inside the worm and is also called (pseudo-) coelomic fluid as it is the fluid in the coelom. It can be obtained by making a hole (punctuation) in the cuticle of the worm and subsequently the fluid will leak out. Alternative the fluid can be drawn out using a needle and syringe. When the BF is obtained from adults it is called adult body fluid (ABF), thus BF also includes helminth adult body fluid (ABF).

4) EV-depleted fractions refer to fractions of the above products that underwent series of centrifugation and ultracentrifugation. The differential centrifugation initiates with e.g. a 30 min centrifugation at 10,000×g where after the supernatant is transferred to polynuclear tubes and ultra-centrifuged at e.g. 110,000×g for 70 min. The supernatant collected after this step is now called EV-depleted fraction due to the pelleting of EVs, and should therefore contain no or few EVs.

5) Extracellular vesicles (EVs) enriched fractions refers to EVs from the above products isolated by a series of centrifugation and ultra-centifugation. The product is e.g. centrifuged at e.g. 10,000×g for 30 min and supernatant is collected and transferred to polynuclear tubes for ultra-centrifugation at e.g. 110,000×g for 70 min. The supernatant (EV-depleted fraction) is collected and the EV pellet is washed in PBS and ultra-centrifuged for again at e.g. 110,000×g for 70 min.

Different widely known methods are available for the purification and enrichment of extracellular vesicles. These include methods such as precipitation or affinity based, size exclusion chromatography, or ultracentrifugation with or without a gradient. All these methods can be utilized for purification of the extracellular vesicles in the present invention.

The helminth product used according to the present disclosure, may thus be selected from fractions, lysates, homogenates, recombinant/synthetic derivates, purified compounds, extracts, body fluids (BF), adult body fluids (ABF), secretion products, EV-depleted fractions, EV-enriched fractions; as well as other extracts and soluble products.

In one embodiment, the helminth product is adult body fluids (ABF).

In another embodiment, the helminth product is an EV-depleted fraction obtained from ABF.

In a third embodiment, the helminth product is an extracellular vesicle-enriched fraction obtained from ABF.

STING/RIG-I Pathway

In one aspect, the invention relates to helminth products capable of inhibiting the activity of the RIG-I/STING pathway, thereby also being capable of regulating the production of type I-III IFNs and proinflammatory cytokines.

The innate immune system relies on several groups of pattern recognition receptors (PRRs) that recognize viral nucleic acids. These PRRs include the sensors cGAS and RIG-I which recognize different nucleic acids in the cytosol. Localization of nucleic acids to the cytosol is associated with tumorigenesis or viral infection, hence, the induction of IFNs by RNA or DNA is essential for defense against virus infections and for anti-cancer immunity. However, excess activation of the RIG-I/STING pathway is associated with clinical conditions involving constitutive activation of IFNs, including monogenic type I interferonopathies and inflammatory or auto-immune disorders.

One way cytosolic nucleic acid is sensed is via the cGAS/STING pathway. STING is a 378 amino acid protein which is expressed broadly in numerous tissue types of both immune and non-immune origin. Cytosolic DNA is recognized by cGAS which upon DNA recognition dimerizes and stimulates the formation of cGAMP. cGAMP then binds directly to STING which triggers phosphorylation/activation of the transcription factor interferon regulatory factor 3 (IRF3) and/or the NF-κB (nuclear factor kappa-light-chain-enhancer of activated B cells) via the TANK-binding kinase 1 (TBK1).

Another pathway involved in sensing cytosolic nucleic acid is the RIG-I/MAVS pathway. RIG-I is part of the RIG-I-like receptor family and contains two N-terminal caspase activation and recruitment domains (CARD). Cytosolic RNA is recognized by RIG-I, inducing a conformational change in the protein, which releases the CARD domain to binding with MAVS. Activation of MAVS results in activation of TBK1 or IκB kinase (IKK) resulting in activation of IRF3 and interferon regulatory factor 7 (IRF7) or NF-κB, respectively.

Although activated by distinct nucleic acids, cGAS-STING and RIG-I-MAVS signaling are functionally interconnected, crosstalk between the nucleic acid sensing mechanisms have been reported as well as involvement of STING in the RIG-I/MAVS pathway. Furthermore, expression levels of RIG-I and STING have been reported to be coordinately regulated through feedback mechanisms. Following their activation, cGAS/STING and RIG-I/MAVS trigger common signaling cascades that ultimately lead to the production of IFNs and proinflammatory cytokines, such as IL-6.

The term “RIG-I/STING pathway” as used herein, refers to the pathway involved in sensing of cytosolic nucleic acids by cGAS or RIG-I and the subsequent activation of STING and/or MAVS to generate downstream signaling cascades, of e.g. IRF3, IRF7 or NF-κB, to produce type I, II, and III IFNs as well as proinflammatory cytokines.

In one embodiment, the helminth product of the present invention may induce interferon production.

In another embodiment, the helminth product may inhibit interferon production.

STING activation may be determined in a number of different ways including the following:

STING activation is induced by STING agonists, such as DNA. STING activation may be determined by determining STING phosphorylation. Thus, it may be preferred that the helminth products are capable of preventing phosphorylation of STING, e.g inducing an at least 2-fold decrease in phosphorylation of STING. Said phosphorylation of STING may in particular be phosphorylation of Ser366 of STING.

Phosphorylation of STING, and particularly phosphorylation of Ser366 of STING may be determined in any useful manner. For example, STING activation may be determined as activation of expression of IFNs or inflammatory cytokines in cells capable of expressing IFNs or cytokines. Examples of such cells include macrophages, dendritic cells, keratinocytes, fibroblasts, monocytes, epithelia cells, B cells, or NK cells. Thus, STING activation may be determined by determining expression of IFNs or cytokines in such cells. Thus, it may be preferred that the helminth products are capable of inhibiting expression of IFNs or cytokines in such cells, e.g. inducing an at least 2-fold decrease in expression of IFNs in such cells, e.g. in macrophages.

In another embodiment, it is preferred that the helminth products may stimulate the described pathways and thereby lead to an increased expression of IFNs or cytokines in the cells, e.g. inducing an at least 2-fold decrease in expression of IFNs and cytokines.

Expression of IFNs or cytokines may be determined by any useful manner, and methods are well-known in the art. For example antibody-based ELISA methods or RT-PCR methods are available and well-known.

STING activation may also be determined as activation of IFNβ promoter activity. Thus, it may be preferred that the helminth products are capable of inhibiting or at least reducing IFNβ promoter activity, e.g. inducing an at least 2-fold decrease in IFNβ promoter activity.

Activity of the IFNβ promoter may for example be determined in recombinant cells comprising a nucleic acid construct encoding a reporter protein under the control of the IFNβ promoter. IFNβ promoter can also be determined in cell free expression systems allowing expression of a reporter protein under the control of the IFNβ promoter.

RIG-I activation may be determined in a number of different ways including the following:

RIG-I activation is induced by RIG-I ligands such as pppRNA or Polyl:C. RIG-I activation may be determined as activation of expression of type I IFN or inflammatory cytokines in cells capable of expressing type I IFN or cytokines. Examples of such cells include macrophages, dendritic cells, keratinocytes, fibroblasts, monocytes, epithelia cells, B cells, or NK cells. Thus, RIG-I activation may be determined by determining expression of IFNs or cytokines in such cells. Thus, it may be preferred that the helminth products are capable of inhibiting expression of IFNs or cytokines in such cells, e.g. inducing an at least 2-fold decrease in expression of IFNs in, e.g. macrophages.

In certain situations, it can be desirable to induce the expression of IFNs or cytokines controlled upstream by RIG-I. In such situations, fractions of the helminth products are able to stimulate expression of IFNs or cytokines in the above mentioned cells e.g. by inducing an at least 2-fold increase in expression of IFNs.

Expression of IFNs or cytokines may be determined by any useful manner, and methods are well-known in the art. For example, antibody-based ELISA methods or RT-PCR methods are available and well-known.

RIG-I activation may also be determined by the phoshorylation level of IRF3 or NF-κB. Thus, it may be preferred that the helminth products are capable of preventing phosphorylation of IRF3 or NF-κB, e.g inducing an at least 2-fold decrease in phosphorylation of IRF3 or NF-κB. In one embodiment, it is preferred that the helminth products are capable of preventing phosphorylation of IRF3, e.g inducing an at least 2-fold decrease in phosphorylation of IRF3.

RIG-I activation may also be determined by the ubiquitination level of RIG-I. Thus, it may be preferred that the helminth products are capable of preventing ubiquitination of RIG-I, e.g inducing an at least 2 fold decrease in ubiquitination of RIG-I.

RIG-I activation may also be determined by the subcellular localization of RIG-I or its interacting partner MAVS. Upon activation, RIG-I interacts with MAVS and co-localizes to the mitochondrial membrane. Thus it may be preferred that the helminth products are capable of preventing localization of RIG-I and MAVS to the mitochondria.

TLR Pathway

The invention generally relates to helminth EV-depleted fractions capable of inhibiting the activity of the TLR pathway, thereby also being capable of regulating the production of type I-III IFNs and proinflammatory cytokines. Furthermore, the invention also related to EV-enriched fractions capable of inducing the activity of the TLR pathway thereby being capable of up-regulating the production of proinflammatory cytokines and IFN-γ (FIG. 4 and FIG. 7).

The activity of the TLR pathway is determined by the same methods utilized for the RIG/STING pathway.

Mechanism of Action of Helminth Products

The helminth products of this disclosure may interact with the RIG-I/STING pathway or the TLR pathway at any step involved in the pathway. The helminth products may thus inhibit or induce the TLR/RIG-I/STING pathway by interacting with any component involved in the TLR/RIG-I/STING pathway. Furthermore, EV fractions of helminth products may also synergize with the TLR/RIG-I/STING pathway by interacting with any component involved in the TLR/RIG-I/STING pathway.

Thus in one embodiment, the helminth products inhibit the TLR/RIG-I/STING pathway by interacting with the pattern recognition receptors (PRRs) of the pathway. In one embodiment, the helminth products inhibit the RIG-I/STING pathway by interacting with cytosolic DNA. In one embodiment, the helminth products inhibit the RIG-I/STING pathway by interacting with cGAS. In one embodiment, the helminth products inhibit the RIG-I/STING pathway by interacting with cGAMP. In one embodiment, the helminth products inhibit the RIG-I/STING pathway by interacting with cytosolic RNA. In one embodiment, the helminth products inhibit the RIG-I/STING pathway by interacting with RIG-I. In one embodiment, such interaction results in degradation of the PRRs.

In one embodiment, the helminth products inhibit the RIG-I/STING pathway by interacting with the components activated by the PPRs. Thus in one embodiment, the helminth products inhibit the RIG-I/STING pathway by interacting with STING. In one embodiment, the helminth products inhibit the RIG-I/STING pathway by interacting with MAVS. In one embodiment, the helminth products inhibit the RIG-I/STING pathway by interacting with cGAMP-STING complex. In one embodiment, the helminth products inhibit the RIG-I/STING pathway by interacting with RIG-I-MAVS complex.

In one embodiment, the helminth products alter the expression level of host molecules involved in the RIG-I/STING pathway. In one embodiment, the helminth products degrade the mRNA of the host molecules involved in the RIG-I/STING pathway. In one embodiment, the helminth products degrade the host molecules involved in the RIG-I/STING pathway. In one embodiment, the helminth products suppress phosphorylation of the host molecules involved in the RIG-I/STING pathway.

In one embodiment, inhibition of the RIG-I/STING pathway results in impairment of the Type I, II and/or III IFN responses.

In one embodiment inhibition of the RIG-I/STING pathway results in impairment of the pro-inflammatory cytokine responses, including but not limited to CXCL10, IL6 and TNF-alpha.

In one embodiment, the helminth products inhibit the TLR pathway by interacting with one or more of the signalling components involved in the TLR pathway.

Clinical Condition Associated with the RIG-I/STING and TLR Pathways

The present invention provides a composition comprising a helminth product for use in preventing, treating and/or ameliorating an inflammatory disorder, cancer or viral infections.

In one embodiment, the composition is provided for use in preventing, treating and/or ameliorating a clinical condition associated with the RIG-I/STING pathway.

In another embodiment, the composition is provided for use in preventing, treating and/or ameliorating a clinical condition associated with TLR pathway.

Several clinical conditions can be associated with both the RIG-I/STING pathway and the TLR pathway.

The RIG-I/STING pathway is involved in inducing type I-III IFN which serves an important role in the immune response to infections. However, various diseases are association with an uncontrolled activation of the RIG-I/STING pathway, such as diseases associated with constitutive activation of IFNs, for example monogenic type I interferonopathies and inflammatory or autoimmune disorders.

In one embodiment, the clinical condition is a disease associated with the RIG-I/STING pathway. The clinical condition may result from an increased activity of the RIG-I/STING pathway.

In one embodiment, the clinical condition is a disease associated with constitutive activation of IFNs.

In one embodiment, the clinical condition is monogenic type I interferonopathy.

The monogenic type I interferonopathy may be selected from the group consisting of: SLE (systemic lupus erythematosus), SAVI (STING associated vasculopathy with onset in infancy), Aicardi-Goutieres Syndrome, Familien Chilblain Lupus, CANDLE (Chronic atypical neutrophilic dermatosis with lipodystrophy and elevated temperature) and JMP (joint contractures, muscle atrophy, microcytic anaemia, and panniculitis-induced lipodystrophy).

In one embodiment, the monogenic type I interferonopathy is selected from the group consisting of: SAVI (STING associated vasculopathy with onset in infancy), Aicardi-Goutieres Syndrome, Familien Chilblain Lupus, CANDLE (Chronic atypical neutrophilic dermatosis with lipodystrophy and elevated temperature) and JMP (joint contractures, muscle atrophy, microcytic anaemia, and panniculitis-induced lipodystrophy).

In one embodiment, the clinical condition is an autoimmune disease.

The autoimmune disease may be selected from the group consisting of psoriasis, psoriatic arthritis, rheumatoid arthritis, Paget's disease, systemic lupus erythematosus (SLE, lupus), Aicardi-Goutieres syndrome, Sjogren's syndrome, Type 1 diabetes, celiac disease and multiple sclerosis.

In one embodiment, the autoimmune disease is selected from the group consisting of psoriatic arthritis, Paget's disease, Aicardi-Goutieres syndrome, Sjogren's syndrome and celiac disease.

In one embodiment, the autoimmune disease is selected from the group consisting of Paget's disease, Aicardi-Goutieres syndrome and Sjogren's syndrome.

In one embodiment, the clinical condition is an inflammatory disease.

The inflammatory disease may be selected from the group consisting of psoriasis, Inflammatory bowel disease (IBD), such as Crohn's disease and ulcerative colitis, allergic rhinitis, asthma, coeliac disease, glomerulonephritis and transplant rejection.

In one embodiment, the inflammatory disease is selected from the group consisting of allergic rhinitis, asthma, coeliac disease, glomerulonephritis and transplant rejection.

In one embodiment, the inflammatory disease is selected from the group consisting of asthma, coeliac disease, glomerulonephritis and transplant rejection.

In one embodiment, the clinical condition is nucleotide-triggered, such as triggered by foreign nucleotides or by host-self nucleotides. In one embodiment, the clinical condition is a nucleotide-triggered inflammation or autoimmune disease. In one embodiment, the clinical condition is DNA triggered. In one embodiment, the clinical condition is RNA triggered.

In one embodiment, the clinical condition is associated with constitutive activation of IFNs resulting from uncontrolled activation of the RIG-I/STING pathway.

In one embodiment, the disease associated with constitutive activation of IFNs is monogenic type I interferonopathy.

The disorder may also be both an inflammatory disorder and an auto-immune disease. Thus, many auto-immune diseases are also inflammatory disorders.

In one embodiment, the composition is used for treatment of cancer.

The present invention is also highly relevant for the treatment of cancer as recent literature (Elion and Cook, 2018) discloses that targeting of the RIG pathway can be lead to future cancer treatments.

In one embodiment, the composition is for use in the treatment of viral infections.

Method of Treatment

In one embodiment, a composition comprising a helminth product for use in preventing, treating and/or ameliorating an inflammatory disorder, cancer or viral infections is provided.

In another embodiment, the composition for use in preventing, treating and/or ameliorating a clinical condition associated with the RIG-I/STING pathway is provided.

The helminth product may be any helminth product as described herein above.

The clinical condition associated with the RIG-I/STING pathway may be any clinical condition as described herein above.

In one embodiment, the prevention, treatment and/or amelioration of said clinical condition associated with the RIG-I/STING pathway is affected by a decrease in the activity of the RIG-I/STING pathway upon administration of a helminth product to an individual in need thereof.

The activity of the RIG-I/STING pathway may be decreased by said helminth product.

As described herein the invention in some embodiments relates to helminth products capable of inhibiting the RIG-I/STING pathway for use in methods of treatment.

In one embodiment, the prevention, treatment and/or amelioration of said clinical condition associated with the RIG-I/STING pathway is affected by an increase in the activity of the RIG-I/STING pathway upon administration of a helminth product to an individual in need thereof.

The activity of the RIG-I/STING pathway may be increased by said helminth product.

As described herein the invention in some embodiments relates to helminth products capable of inducing the RIG-I/STING pathway for use in methods of treatment.

In another embodiment, a composition comprising a helminth product for use in preventing, treating and/or ameliorating a clinical condition associated with the TLR pathway is provided.

The helminth product may be any helminth product as described herein above.

In one embodiment, the prevention, treatment and/or amelioration of said clinical condition associated with the TLR pathway is affected by a decrease in the activity of the TLR pathway upon administration of a helminth product to an individual in need thereof.

The activity of the TLR pathway may be decreased by said helminth product.

As described herein the invention in some embodiments relates to helminth products capable of inhibiting the TLR pathway for use in methods of treatment.

In one embodiment, the prevention, treatment and/or amelioration of said clinical condition associated with the TLR pathway is affected by an increase in the activity of the TLR pathway upon administration of a helminth product to an individual in need thereof.

The activity of the TLR pathway may be increased by said helminth product. Thus, in one embodiment, a method of inducing the TLR pathway in an individual in need thereof is provided, the method comprising administering a therapeutically effective amount of a helminth product to said individual.

As described herein the invention in some embodiments relates to helminth products capable of inducing the TLR pathway for use in methods of treatment.

Whilst it is possible for the helminth products of the present invention to be administered as the raw product, it is preferred to present them in the form of a pharmaceutical formulation. Accordingly, the present invention further provides a pharmaceutical formulation, which comprises a helminth product of the present disclosure and a pharmaceutically acceptable carrier therefore. The invention also provides pharmaceutical formulations comprising a helminth product of the disclosure and a pharmaceutically acceptable carrier therefore.

The pharmaceutical formulations may be prepared by conventional techniques, e.g. as described in Remington: The Science and Practice of Pharmacy 2005, Lippincott, Williams & Wilkins.

The pharmaceutically acceptable carriers can be either solid or liquid. Solid form preparations include powders, tablets, pills, capsules, cachets, suppositories, and dispersible granules. A solid carrier can be one or more excipients, which may also act as diluents, flavoring agents, solubilizers, lubricants, suspending agents, binders, preservatives, wetting agents, tablet disintegrating agents, or an encapsulating material.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The helminth products of the present invention may be formulated for parenteral administration and may be presented in unit dose form in ampoules, pre filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

Preferably, the formulation will comprise about 0.5% to 75% by weight of the active ingredient(s) with the remainder consisting of suitable pharmaceutical excipients as described herein.

The helminth products of the invention are in general administered in an “effective amount” or an amount necessary to achieve an “effective level” in the individual patient. When the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the patient that corresponds to a concentration of the helminth product according to the invention.

The helminth products of the invention may be administered together with one or more other active compounds, typically with one or more other active compounds useful for treatment of the particular disorder to be treated.

The composition may be administered to a patient in need of such treatment at various sites, for example administration at sites which bypass absorption, such as in an artery or vein or in the brain, and at sites which involve absorption, such as in the skin, under the skin, in a muscle or in the abdomen.

### EXAMPLES

### Example 1: Preparation of Materials and Methods

Cell Culture

The human acute monocytic leukaemia cell line (THP-1) was cultured in RPMI 1640 (Lonza) supplemented with 10% heat inactivated fetal calf serum, 200 U/mI Penicillin, 100 mg/mI Streptomycin and 600 mg/mI glutamine (hereafter termed RPMI complete). To differentiate THP-1 cells into adherent phenotypically macrophages, cells were stimulated with 100 nM Phorbol 12-myristate 13-acetate (PMA, Sigma Aldrich 79346) in RPMI complete for 24 h before medium was refreshed with normal RPMI complete and allowed to further differentiate an additional day (hereafter defined as macrophages).

Peripheral Blood Mononuclear cells (PBMCs) were isolated from healthy donors by Ficoll Paque gradient centrifugation (GE Healthcare).

Differentiation of monocyte-derived macrophages (MdMs) was performed by separating monocytes from PBMCs by adherence to plastic in RPMI 1640 supplemented with 10% heat inactivated AB-positive human serum (Sigma-Aldrich). Differentiation of monocytes to macrophages was achieved by culturing in Dulbecco's

Modified Eagle Medium (DMEM) supplemented with 10% heat inactivated AB-positive human serum, 200 IIU/ml Penicillin, 100 μg/ml Streptomycin, 600 μg/ml glutamine for 10 days in the presence of 10 ng/ml M-CSF (R&D systems).

Preparation of Ascaris suum Adult Body Fluid (ABF)

Ascaris suum ABF was obtained from freshly collected adult worms by decapitation and collection of the pseudocoelomic fluid followed by centrifugation at 10,000 g for 15 min. The supernatant was stored at −80° C. before use.

Purification of EV (Extracellular Vesicle)-Fraction and EV-Free-Fraction

ABF underwent differential centrifugations initiated with 30 min centrifugation at 10,000×g in order to discard cellular debris.

The supernatant was transferred to polynuclear tubes (Beckman Coulter) and ultra-centrifuged at 110,000×g for 70 min in a Beckman Coulter Optima L-80 Ultracentrifuge using a Type 50 TI Fixed Angle Rotor. The supernatant (ABF extracellular vesicle-depleted fraction) was collected and stored at −80° C. and EV pellet was then washed in PBS and ultracentrifuged for another 70 min at 110,000×g. Finally, the EVs were suspended in 180 μl PBS and stored at −80° C. until further analysis. The presence of EVs in the sample was confirmed by Nanosight (Nanopaticle Tracking Analysis (NTA)). The final dissolved EV pellet is termed the extracellular vesicle-enriched fraction.

Preparation of Worm Products

ABF and ABF extracellular vesicle-depleted fraction (EV-depleted) and the extracellular-enriched fraction were added to the PBMCs and incubated for 30 min before the addition of stimulations (DNA or LPS) and a further incubation at 37° C. for 20 hours. ABF and the extracellular vesicle-depleted fraction (EV-depleted) were measured by BCA protein Assay (Thermo Fischer Scientific) by methods known by those of skill in the art.

The number of EVs in the extracellular-enriched fraction corresponded to the number of extracellular vesicles in the ABF, i.e. EVs150 includes the number of vesicles found in 150 ug/ml ABF.

Stimulation

Standard stimulation of isolated PBMCs or monocyte-derived macrophages (MdMs) with dsDNA (0.1 μg/well) was conducted on 1×105 cells in a 96-well (PBMCs) or 1.5×10⁵ cells in 24-well plate (MdMs) format using Lipofectamine 2000 (Life Technologies 11668-019) as carrier. Transfection protocols were as according to the manufacturer's instructions using a ratio of Lipofectamine to DNA of 1:1.

Bacterial lipopolysaccharide was added to the medium at 10 ng/ml concentration in the same format as described with dsDNA but without a carrier. 20 hours post stimulation supernatants were harvested and kept at −20° C. until cytokine and IFN quantification.

U-Plex (Mesocale)

Protein levels of cytokines and interferons (type I, II and III) in supernatant were evaluated using U-plex Biomarker Group 1 (Human) multiplex assays (Mesoscale) following the manufactures instructions.

Measurement of Functional Type I IFN

Bioactive functional type I IFN was quantified in supernatants using the reporter cell line HEK-Blue™ IFN-α/β (Invivogen). The cell line was maintained in DMEM+5 GlutaMax™-I (Gibco®, Life Technologies), supplemented with 10% heat-inactivated FCS, 100 μg/mL Streptomycin and 200 U/mL Penicillin, 100 μg/mL Normocin (InvivoGen), 30 μg/mL Blasticidin (InvivoGen) and 100 μg/mL eocin (InvivoGen). Cells were passaged using 1× trypsin (Gibco®, Life Technologies). For measurement of functional type IFN, cells were seeded at 3×10⁴ cells/well in 96-well plates in 150 μL medium. Cells were grown as previously described, but without blasticidin and zeocin. The following day, 50 μL of supernatant from stimulated cells or a type I IFN standard range was added to the cells. After 24 hours of incubation, 20 μL of supernatant from the HEK-Blue cells were subsequently added to 180 μL QUANTI-Blue™. SEAP activity was assessed by measuring optical density (OD) at 620 nm on a microplate reader (ELx808, BioTEK). The standard range was made with IFN-α (IFNa2 PBL Assay Science) and ranged from 2 to 500 U/mL.

Enzyme-Linked Immunosorbent Assay (ELISA)

Protein levels of IL-6 in supernatants were evaluated using ELISA kits (R&D systems) following the manufacturer's instructions.

Data Presentation

All data were plotted using Graph Pad Prism 7.0 (GraphPad Software, San Diego, Calif., USA). Data presented are expressed as means±standard error of mean (±SEM).

### Example 2: Treatment of Differentiated THP-1 Cells Using Varying Amounts of A. suum ABF

Method

Human acute monocytic leukaemia cell line (THP-1 cells) were differentiated into adherent macrophage-like-cells for 48 hours by stimulation with Phorbol 12-myristate 13-acetate. These cells were then pre-treated with Ascaris suum adult body fluid (ABF) for 30 min before removing the media and washing the cells with PBS to ensure all ABF was removed. The amount of ABF used for treatment was determined via the protein-content of the ABF, measured by Nanodrop©. In this experiment either 600 μg protein per 5×10⁴ cells or 350 μg per 5×10⁴ cells of ABF were used.

After washing, the cells were stimulated with DNA (0.5 μg/5×10⁴ cells) or bacterial lipopolysaccharide (LPS) (200 ng/ml) for 24 hours. At that time-point supernatants were collected and measured for amount of released Interleukin-6 (IL-6) by ELISA as well as released type 1 interferon (IFN) via HEK blue bioassay.

Results

The example demonstrates that upon treatment with Ascaris suum ABF, the IFN-I production in response to DNA, a STING agonist, by differentiated THP-1 cells is strongly decreased (FIG. 2a) in a concentration dependent manner.

The example further demonstrates that treatment of differentiated THP-1 cells with Ascaris suum ABF strongly impairs the IL-6 production in response to LPS, a TLR-4 ligand, in a concentration dependent manner (FIG. 2b).

### Example 3: Treatment of Differentiated THP-1 Cells with A. suum ABF at Varying Time Periods

Method

Human acute monocytic leukaemia cell line (THP-1 cells) were differentiated into adherent macrophage-like-cells for 48 hours by stimulation with Phorbol 12-myristate 13-acetate. These cells were then pre-treated with Ascaris suum adult body fluid (ABF) for 30 min or 4 h before removing the media and washing the cells with PBS to ensure all ABF was removed. After washing, the cells were stimulated with DNA (0.5 μg/5×10⁴ cells) or bacterial lipopolysaccharide (LPS) (200 ng/ml) for 24 hours. At that time-point supernatants were collected and measured for amount of released Interleukin-6 (IL-6) by ELISA as well as released type 1 interferon (IFN) via HEK blue bioassay.

Results

The example demonstrates that already after a treatment duration of 30 min with ABF the IFN-I production in response to DNA, a STING agonist, by differentiated THP-1 cells is strongly decreased (FIG. 3a).

This example further demonstrates that a 30 min treatment of differentiated THP-1 cells with ABF strongly impairs the IL-6 production in response to LPS, a TLR-4 ligand (FIG. 3b).

### Example 4: Treatment of Differentiated THP-1 Cells with Different Fractions of A. suum ABF

Method

Human acute monocytic leukaemia cell line (THP-1 cells) were differentiated into adherent macrophage-like-cells for 48 hours by stimulation with Phorbol 12-myristate 13-acetate. These cells were then pre-treated with different Ascaris suum adult body fluid (ABF) fractions for 30 min. The different fractions were produced by using a series of centrifugation and ultra-centrifugation steps as described in example 1. The medium was then removed and the cells washed with PBS to ensure all ABF was removed. After washing, the cells were stimulated with DNA (0.5 μg/5×10⁴ cells) or bacterial lipopolysaccharide (LPS) (200 ng/ml) for 24 hours. At that time-point supernatants were collected and measured for amount of released Interleukin-6 (IL-6) by ELISA.

Results

The example demonstrates that the different ABF fractions have different effects on the IL-6 production of differentiated THP-1 cells. The extracellular vesicle-enriched fraction increases the IL-6 response to LPS, a TLR-4 ligand, and even to DNA, a STING agonist (FIG. 4). This example further demonstrates that the extracellular vesicle-depleted fraction decreases the IL-6 response after LPS stimulation (FIG. 4).

### Example 5: Treatment of PBMCs Using Varying Amounts of ABF and Different Fractions of A. suum ABF

Methods

Peripheral Blood Mononuclear cells (PBMCs) were isolated from healthy donors by Ficoll Paque gradient centrifugation (GE Healthcare).

Isolated PBMCs were pre-treated with Ascaris suum adult body fluid (ABF), an extracellular vesicle-enriched fraction or an extracellular vesicle-depleted fraction for 30 min. The EV-enriched and EV-depleted fraction were produced using a series of centrifugation and ultra-centrifugation steps as described in example 1. The amount of A. suum ABF and extracellular vesicle-depleted fraction used for treatment was determined via the protein-content by BCA protein Assay (Thermo Fischer Scientific). The cells were then stimulated with DNA (0.1 μg/1×105 cells) or bacterial lipopolysaccharide (LPS) (10 ng/ml) for 20 hours. At that time-point supernatants were collected and measured for amount of released Interleukin-6 (IL-6), Tumor necrosis factor alpha (TNF-alpha), as well as released type I, II and III IFNs by multiplex ELISA (U-plex MSD mesoscale).

Results

The example demonstrates that treatment with Ascaris suum ABF strongly impairs the production of IL-6 and TNFa in response to LPS, a TLR-4 ligand, in a concentration dependent manner (FIG. 5A+B). The extracellular vesicle-depleted fraction show the same tendency with decreasing production of IL-6 and TNFa in response to LPS in a concentration dependent manner (FIG. 5A+B).

The example also demonstrates that upon treatment with Ascaris suum ABF and the extracellular vesicle-depleted fraction, the type-I and type-III IFN (IFN-α/β and -λ, respectively) and CXCL10/IP-10 production in response to DNA (STING agonist), are strongly decreased (FIG. 5C and FIG. 6A-C) in a concentration dependent manner. Production of type-II IFN (IFN-γ) increased only upon treatment with ABF (FIG. 6D).

This example further demonstrates that the extracellular vesicle-enriched fraction have a different effect on pro-inflammatory cytokine production than ABF and the extracellular vesicle-depleted fraction. Treatment with the extracellular vesicle-enriched fraction alone increases production of IL-6 and TNFa upon LPS stimulation as well as upon DNA stimulation (FIG. 7A+B). The CXCL10/IP-10 production after treatment with extracellular vesicle-enriched fraction is however not affected by LPS stimulation and slightly decreases upon DNA stimulation (FIG. 7C), but not in a dose dependent manner. Next, the example demonstrates the interferon responses upon treatment with the extracellular vesicle-enriched fraction have no significant enhancement or inhibitory effects for type I and III IFNs following DNA stimulation. However, for IFN-γ a significant and synergistic effects of extracellular vesicle-enriched fraction treatment in a dose dependent manner is observed (FIG. 8A-D).

### Example 6: Treatment of Differentiated Monocyte-Derived Macrophages with Different Fractions of A. suum ABF

Methods

PBMCs were isolated from healthy donors by Ficoll Paque gradient centrifugation (GE Healthcare). Monocytes were separated from PBMCs by adherence to plastic in RPMI 1640 supplemented with 10% heat inactivated AB-positive human serum (Sigma-Aldrich). Differentiation of monocytes to macrophages was achieved by culturing in Dulbecco's Modified Eagle Medium (DMEM) supplemented with 10% heat inactivated AB-positive human serum, 200 IIU/ml Penicillin, 100 μg/ml Streptomycin, 600 μg/ml glutamine for 10 days in the presence of 10 ng/ml M-CSF (R&D systems). Monocyte derived macrophages (MdMs) were pre-treated with Ascaris suum adult body fluid (ABF), the extracellular vesicle-enriched fraction or the extracellular vesicle-depleted fraction for 30 min. The extracellular vesicle-enriched fraction and the extracellular vesicle-enriched fraction were produced by using a series of centrifugation and ultra-centrifugation steps as described in example 1. The amount of A. suum ABF and the extracellular vesicle-depleted fraction used for treatment was determined via the protein-content by BCA protein Assay (Thermo Fischer Scientific). The cells were then stimulated with DNA (0.1 μg/1.5×105 cells) for 20 hours. At that time-point supernatants were collected and measured for amounts of released CXCL10/IP-10 by ELISA and production of Interleukin-6 (IL-6), Tumor necrosis factor alpha (TNFa), as well as released type I, II and III IFNs by multiplex ELISA (U-plex MSD mesoscale).

Results

The example demonstrates that the different ABF fractions have different effects on IL-6 and TNFa production in MDMs. Importantly, ABF and the extracellular vesicle-enriched fraction increases IL-6 and TNFa in response to DNA (STING agonist) (FIG. 9A+B), which support earlier data obtained from THP-1 cells (FIG. 4). Treatment of MDMs with ABF, but not the extracellular vesicle-enriched fraction, reduces CXCL10/IP-10 response upon stimulation with DNA (FIG. 9C). Next, the example demonstrates that upon treatment with the extracellular vesicle-depleted fraction, the production of IL6 and TNF-alpha is not affected. However, the extracellular vesicle-depleted fraction do impair CXCL10/IP-10 responses (FIG. 9A-C).

The example further demonstrates that ABF and the extracellular vesicle-depleted fraction are able to suppress IFN-β and IFN-λ, in MDMs upon stimulation with DNA (FIG. 10B-C). In contrary, no effects are observed for IFN-γ (FIG. 10D). In addition, the extracellular vesicle-enriched fraction do not have any effects in MDMs on type I, II and III IFNs (FIG. 10A-D).

1-57. (canceled)

58. A method of preventing, treating and/or ameliorating a clinical condition associated with the RIG-I/STING pathway, the method comprising administering a helminth product to an individual in need thereof, wherein the helminth product comprises a lysate or secretion of an Ascaridida worm and/or egg.

59. The method according to claim 58, wherein the Ascaridida worm is of the Ascaridoidea superfamily

60. The method according to claim 58, wherein the Ascaridoidea is of the Ascaris genus.

61. The method according to claim 58, wherein the lysate is configured as obtained by decapitation of the Ascaridida worm.

62. The method according to claim 58, wherein the lysate is Ascaridida worm body fluid (pseudocoelomic fluid).

63. The method according to claim 58, wherein the Ascaridida worm body fluid is adult body fluid.

64. The method according to claim 58, wherein the lysate or secretion has been fractionated into at least two fractions.

65. The method according to claim 64, wherein at least one of said at least two fractions comprises an extracellular vesicle-depleted fraction.

66. The method according to claim 64, wherein at least one of said at least two fractions is obtained by a centrifugation procedure.

67. The method according to claim 58, wherein the activity of the RIG-I/STING pathway is decreased.

68. The method according to claim 58, wherein an activity of an immune system of said individual is inhibited by said administering to prevent, treat or ameliorate an inflammatory disorder, such as an autoimmune disease or disorder.

69. The method according to claim 68, wherein the inflammatory disorder is selected from the group consisting of diseases associated with constitutive activation of Interferons (including type I, II and III IFNs), monogenic type I interferonopathies, autoimmune diseases and inflammatory diseases.

70. The method according to claim 69, wherein the monogenic type I interferonopathy is selected from the group consisting of SAVI (STING associated vasculopathy with onset in infancy), Aicardi-Goutieres Syndrome, Familial Chilblain Lupus, CANDLE (Chronic atypical neutrophilic dermatosis with lipodystrophy and elevated temperature) and JMP (joint contractures, muscle atrophy, microcytic anaemia, and panniculitis-induced lipodystrophy).

71. The method according to claim 68, wherein said disorder is a nucleotide-triggered, such as DNA-triggered or RNA-triggered inflammatory disorder.

72. A method of manufacturing a helminth product comprising an extracellular vesicle-depleted fraction, the method comprising:
a. providing Ascaridida worms and/or eggs,
b. lysing the Ascaridida worms and/or eggs of a), thus providing a lysate such as worm body fluid, and
c. fractionating the lysate of b) to generate an extracellular vesicle-depleted fraction.

73. The method of manufacturing according to claim 72, wherein the worm body fluid is extracted from adults worms and therefore is Adult Body Fluid (ABF).

74. The method of manufacturing according to claim 73, wherein the worm body fluid is subjected to a centrifugation procedure to obtain a supernatant defined as an extracellular-depleted fraction and a extracellular vesicle fraction, which is defined as an extracellular vesicle-enriched fraction.

75. The method of manufacturing according to claim 74, wherein the centrifugation procedure is performed using the following procedure:
d. centrifuging the extracted fluid or medium solution to discard cellular debris and obtain a supernatant,
e. further centrifuging said supernatant to obtain an extracellular vesicle pellet and an extracellular vesicle-depleted fraction.

76. The method of manufacturing according to claim 75, wherein the centrifugation procedure is performed as a three-step procedure where each centrifugation step is conducted at least at 5,000×g.

77. A helminth product obtainable by the method of manufacturing according to claim 72.
